(19) United States
(12) Patent Application Publication
(10) Pub. No.: US 2015/0087048 A1
(43) Pub. Date: Mar. 26, 2015

(54) APPARATUS AND METHOD OF PROCESSING MICROORGANISMS

(71) Applicants: Marcus Brian Mayhall Fenton, St. Neots (GB); Olga Koroleva, Godmanchester (GB); Michelle Gina Elizabeth Gothard, Royston (GB); Christopher Drake, Letchworth (GB)

(72) Inventors: Marcus Brian Mayhall Fenton, St. Neots (GB); Olga Koroleva, Godmanchester (GB); Michelle Gina Elizabeth Gothard, Royston (GB); Christopher Drake, Letchworth (GB)

(21) Appl. No.: 14/124,203

(22) PCT Filed: Jun. 22, 2012
(86) PCT No.: PCT/GB2012/051476

§ 371 Date: Nov. 17, 2014

(30) Foreign Application Priority Data
Jun. 22, 2011 (GB) 1110575.6

(51) Int. Cl. C12N 1/12

(52) U.S. Cl. CPC C12N 1/12
USPC 435/257.1; 435/306.1

(57) ABSTRACT

A method and apparatus for processing microorganisms is provided. The method comprises mixing microorganisms with a working fluid to form an working fluid slurry, and injecting a transport fluid through a transport fluid nozzle into the working fluid slurry in order to disrupt the cellular structure of the microorganisms.

The present invention is directed to an apparatus and related method of processing algae or similar microbial species and microorganisms. More specifically, the apparatus and method of the present invention are concerned with recovering intracellular materials and components contained in the microorganisms that are of economic value, and/or processing the microorganisms for a subsequent use or application.

Biological oil-bearing organisms such as photosynthetic, heterotrophic and mixotrophic protists, yeast and cyanobacteria (also known as blue green algae, BGA) have been found to contain oils which have both the quantity and compositional profile which make them suitable for conversion into liquid fuels, such as jet fuel and diesel. For all of these organisms, photosynthetic conversion of sunlight is typically 8-10% compared to 3-5% for higher plants with oil-bearing structures, such as oil seed rape. The oil is produced in these organisms as a food store and is often produced in response to environmental or physiological stress. Oil content in some species can be as high as 50% by volume. Some of these organisms also contain other compounds of chemical and pharmaceutical interest increasing their potential economic value. Examples of such compounds are the oil-soluble pigments lycopene and beta-carotene that are produced by certain types of algae, as well as other compounds such as lipids, vitamins, pigments, pharmacological compounds and oils.

In some circumstances the biological organisms such as microalgae, photosynthetic protists, diatoms and cyanobacteria are grown as a means of producing commercially useful cellular contents which are of significant economic value in the manufacture of products such as cosmetics, pharmaceuticals, foods, nutritional supplements, pigments, for example. In addition, these microorganisms can convert sugars derived from lignocellulosic materials into oils, ethanol and lactic acid.

These organisms can exhibit rapid growth rates with multiple generations propagated over hours or days, and could be cultured on land that is not suitable for agriculture or housing. Also in the favour of these organisms is the possibility of using CO₂ captured from other industrial processes as a carbon feedstock for photosynthesis and growth. In some cases the use of grey water/municipal sewage as part of the growth medium may be a serious consideration.

As well as the extraction of intracellular contents for commercial use, in some industries the entire cell or the cell wall component may constitute the target material for the process. For example, water companies are now looking to use microalgae as a form of bioremediation. The algae are grown in the ‘clean’ water streams from effluent processing sites as a way of stripping excess nitrates and phosphates from the water prior to discharge into a watercourse. The algae are harvested from the water prior to discharge and can be prepared as feedstock for methane (Biogas) generation via anaerobic digestion (bioreactors). To increase the efficiency of bioreactors it is advantageous to disrupt the cellular structure of the algae in advance, releasing nutrients and increasing effective surface area for the anaerobes in the bioreactor to access.

Despite their suitability in their role as producers of commercially useful compounds and materials there are serious challenges to overcome in making the use of these organisms into a viable economic proposition. For example, in the field of utilising micro-algae for oil production, the processes and technologies involved in the processing of algae are divided into “upstream” and “downstream” areas. Upstream processes refer to the selection of appropriate oil-rich species and their cultures. Downstream processes refer to those activities and technologies involved in separating the oil, proteins and other valuable products from the remaining compounds of these organisms. At present the industry considers the downstream phase as a two step process, consisting of a pre-treatment to weaken the cellular structure of the organism, followed by drying and concentration of the resultant biomass and then cold pressing. Oil recovery using this method is energy intensive and far from optimal with figures being quoted as low as 30-40% recovery of the total present in the biomass. Also the oil is usually extracted from the pressed biomass via solvents, e.g. hexane, raising both environmental and economic questions of its efficacy.

As well as cold pressing of oil-bearing biomass, two other approaches which have been promoted for processing all sorts of algae in a variety of industries are ultra-sonication and explosive decompression. Both techniques seek to disrupt the outer wall of the organism in order to release the cell contents containing the oil or, where full disruption is not not desired or possible, these techniques aim to increase the porosity of the cell wall to aid in extraction of useful compounds, possibly via further chemical or enzymatic processing. Because of the small size of the cells in the species of interest (typically 3-100 μm), and the complex cell wall compositions, effective disruption is very difficult. Ultra-sonication utilises the energy from high frequency sound waves to generate tiny cavitation bubbles in the liquid medium around the cells creating localised shear. As a small scale batch process this can be very efficient, but on a large scale the energy input is high, and the disruptive efficiency in high throughput continuous flow processes is very poor. Explosive decompression utilizes the solubility of CO₂ or Nitrogen in water under pressure. The biomass for treatment is placed in a pressure vessel into which CO₂ or Nitrogen is introduced under pressure. The increased pressure allows the gas to solvate in the water phase both inside and outside of the microbial cells. When the pressure is suddenly released the gas in solution rapidly tries to reach its new solution equilibrium and rapidly boils out of solution. The massive volume expansion of the gas rips the cells apart. Like ultra-sonication, explosive decompression can only be applied in a batch process and thus does not lend itself to current refinery processes.

It is an aim of the present invention to obviate or mitigate one or more of the aforementioned disadvantages with these existing processing apparatus and methods.

According to a first aspect of the present invention, there is provided a method of processing microorganisms, the method comprising:
mixing microorganisms with a working fluid to form a working fluid slurry; and
injecting a transport fluid through a transport fluid nozzle into the working fluid slurry in order to disrupt the cellular structure of the microorganisms.

The microorganisms may be algae. References to “algae” in this specification should be understood to be references to any aquatic photosynthetic, heterotrophic or mixotrophic organism.

The method may further comprise the steps of:
supplying the working fluid slurry to a fluid processor passage having an inlet and an outlet, wherein the cross sectional area of the passage between the inlet and the outlet does not reduce below the cross sectional area at the inlet;
supplying the transport fluid from a transport fluid source to a transport fluid nozzle which circumscribes the passage and opens into the passage intermediate the inlet and the outlet, the transport fluid nozzle having a nozzle inlet, a nozzle outlet, and a nozzle throat intermediate the nozzle inlet and nozzle outlet which has a cross sectional area which is less than that of both the nozzle inlet and nozzle outlet; and
accelerating the transport fluid through the transport fluid nozzle so as to inject the transport fluid into the working fluid slurry.

The method may further comprise the step of recovering any intracellular material released by the microorganisms downstream of the fluid processor.

The intracellular material may be oil. The intracellular material may alternatively be one or more of the group comprising oil, protein, pigments, carbohydrates, pharmalogical or other metabolites, and other chemical and pharmaceutical compounds, such as glycerol.

The recovery step may include separating the intracellular material from the working fluid slurry in a separation vessel.

The recovery step may include adding an additive to the working fluid slurry to encourage the release of the intracellular material. The additive may include a flocculant for the concentration and separation of the material within the microorganisms from the rest of the working fluid.

The recovery step may include adding demulsifiers to the working fluid slurry to facilitate separation of the oil fraction from the aqueous fraction.

The working fluid may be water. The water may have a salt content of between 1 and 50 per mille.

The working fluid may be selected from a group of working fluids comprising organic solvents such as hexane, n-methyl morpholine n-oxide, dodecane, dichloromethane, chloroform, ethanol and other solvents such as dimethyl sulfoxide.

The mixing step may include the addition of one or more degrading additives to chemically degrade the cellular structure of the microorganisms. One degrading additive may be enzymes to enzymatically degrade the cellular structure of the microorganisms. One or more pH-altering additives may also be added during the mixing step to alter the pH of the working fluid slurry.

The transport fluid may be steam and the transport fluid source may be a steam generator.

The method may further comprise the steps of:
injecting a compressed gas into the working fluid slurry prior to the step of supplying the fluid to the fluid processor passage; and
holding the working fluid slurry under pressure upstream of the fluid processor.

The compressed gas may be carbon dioxide. Alternatively, the compressed gas may be nitrogen or air.

The working fluid slurry may be supplied via an entrainment port which opens into the passage downstream of the nozzle outlet.

The method may further comprise the step of supplying a process fluid to the inlet of the passage. The process fluid may be water. The water may have a salt content of between 1 and 50 per mille. Alternatively, the process fluid may be selected from a group of working fluids comprising hexane, decane, dichloromethane, n-methyl morpholine n-oxide, chloroform, ethanol, organic solvents, and organosulphur compounds such as dimethyl sulfoxide.

The process fluid and working fluid slurry may have different osmotic potentials and/or temperatures.

The supply and subsequent injection of the transport fluid may be pulsed.

The method may further comprise the step of returning fluid flow from downstream of the passage outlet to the inlet of the passage via a return loop and diverter valve.

The method may further comprise the step of returning fluid flow from downstream of the passage outlet to a growth vessel via a return loop and diverter valve. The working fluid slurry returned to the growth vessel may contain live microorganisms.

According to a second aspect of the present invention, there is provided an apparatus for processing microorganisms, the apparatus comprising:
a mixing vessel adapted to receive and mix supplies of microorganisms and a working fluid to form a working fluid slurry; and
a transport fluid nozzle adapted to inject a transport fluid into the working fluid slurry in order disrupt the cellular structure of the microorganisms.

The apparatus may further comprise:
a fluid processor including the transport fluid nozzle and a passage having an inlet and an outlet; and
a transport fluid source in fluid communication with the transport fluid nozzle;
wherein the transport fluid nozzle circumscribes the passage and opens into the passage intermediate the inlet and outlet, the mixing vessel is in fluid communication with the passage, the cross sectional area of the passage between the inlet and outlet does not reduce below the cross sectional area at the inlet, and the transport fluid nozzle is a convergent-divergent nozzle having a nozzle inlet, a nozzle throat, and a nozzle outlet, and the cross sectional area of the nozzle throat is less than that of both the nozzle inlet and nozzle outlet.

The apparatus may further comprise a first control valve adapted to control flow of the working fluid slurry from the mixing vessel to the passage.

The mixing vessel may be in fluid communication with the inlet of the passage. Alternatively, the processor may further comprise an entrainment port opening into the passage downstream of the nozzle outlet, wherein the mixing vessel is in fluid communication with the entrainment port.

The transport fluid source may be a steam generator. A second control valve may control flow of transport fluid from the transport fluid source to the transport fluid nozzle.

The transport fluid source may include a transport fluid pressure controller.

The transport fluid source may be adapted so as to pulse the supply of transport fluid.

The fluid processor may further comprise an additive port in fluid communication with the passage. The additive port may be immediately downstream of the transport fluid nozzle outlet.

The apparatus may comprise a plurality of fluid processors connected to one another in series and/or parallel.

The apparatus may further comprise a separation vessel in fluid communication with the outlet of the passage. The separation vessel may comprise a centrifuge.

The transport fluid nozzle may have an equivalent angle of expansion from the nozzle throat to nozzle outlet of between 8 and 30 degrees.

The fluid processor may include a housing and a protrusion which extends axially into the housing, whereby the protrusion defines a portion of the passage downstream of the passage inlet and an inner surface of the transport fluid nozzle outlet.

The passage has a longitudinal axis, and the inner surface of the transport fluid nozzle outlet may be at a maximum angle of 70 degrees relative to the longitudinal axis. Preferably, the inner surface of the transport fluid nozzle outlet is at an angle of between 15 and 35 degrees relative to the longitudinal axis.

The apparatus may further comprise a pump adapted to pump working fluid slurry into the fluid processor passage. The pump may be a progressive cavity pump.

The apparatus may further comprise a first return loop and diverter valve downstream of the passage outlet, the first return loop and diverter valve adapted to return fluid flow to the inlet of the passage.

The apparatus may further comprise a growth vessel, and a second return loop and diverter valve adapted to return fluid flow from the processing vessel to the growth vessel. The second return loop may divert the working fluid slurry downstream of the passage outlet, back to the growth container.

The mixing vessel may comprise a gas injector adapted to inject a compressed gas into the vessel. The apparatus may further comprise a first pressure regulating valve adapted to maintain a predetermined pressure upstream of the fluid processor. The apparatus may further comprise a second pressure regulating valve adapted to maintain a predetermined pressure downstream of the fluid processor.

The apparatus may further comprise one or more flow control valves and a programmable system controller adapted to selectively activate the one or more control valves.

A preferred embodiment of the present invention will now be described, by way of example only, with reference to the accompanying drawings, in which:

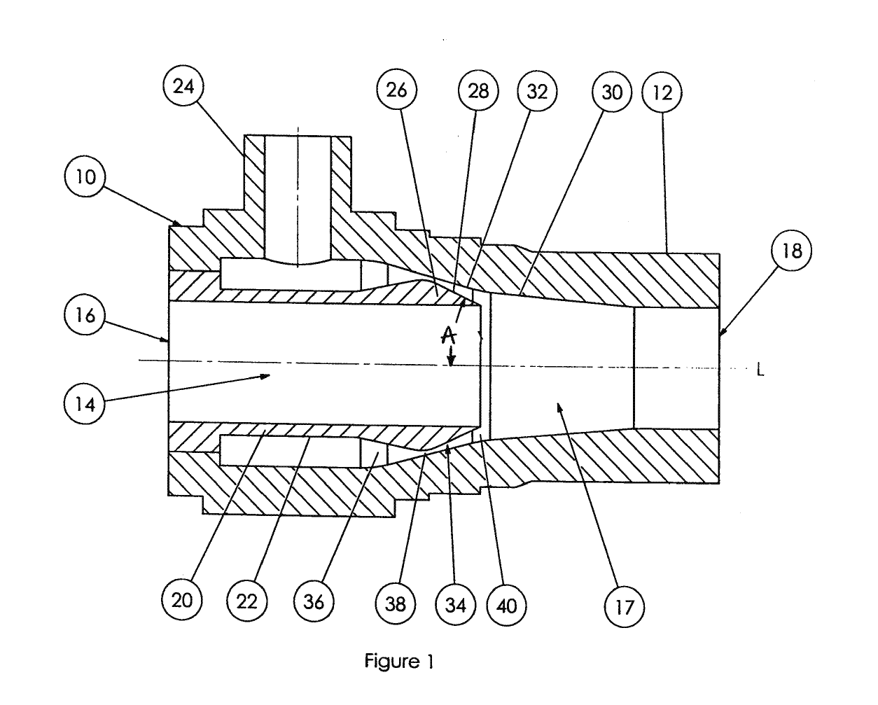

FIG. 1 is a cross sectional view of a fluid processor;

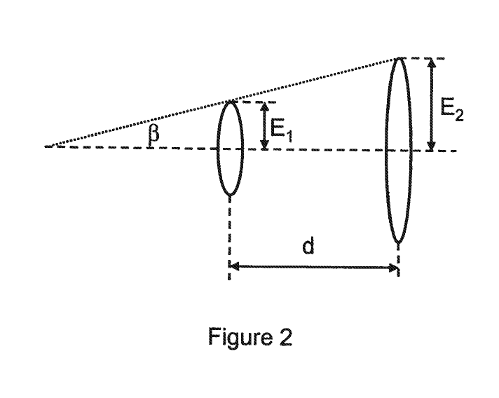

FIG. 2 is a diagram allowing the expansion angle of a transport fluid nozzle in the fluid processor to be calculated;

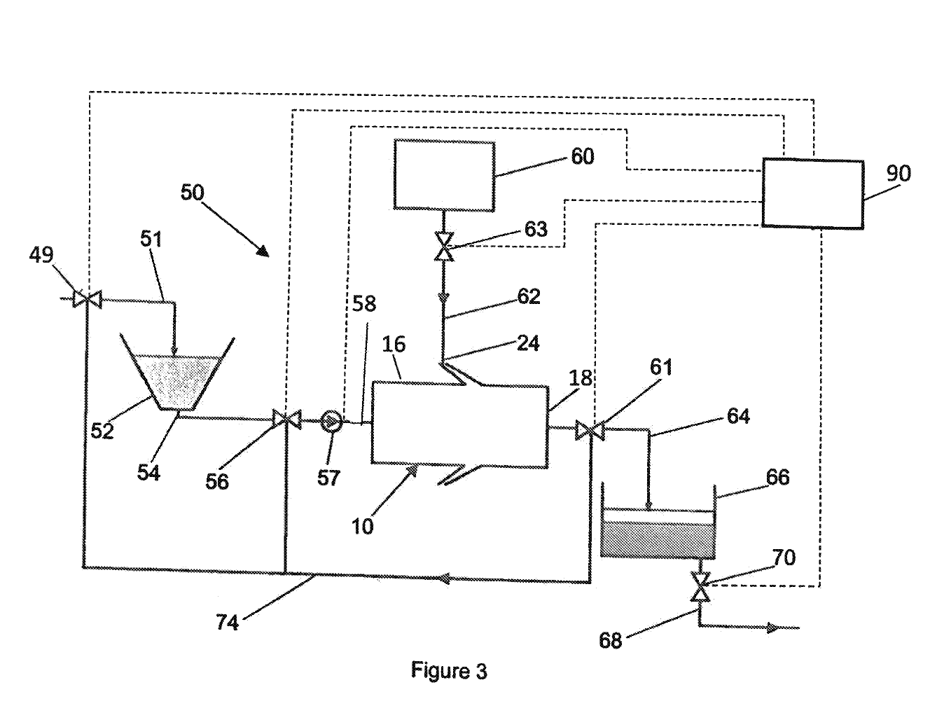

FIG. 3 is a schematic view of an apparatus for the processing of microorganisms;

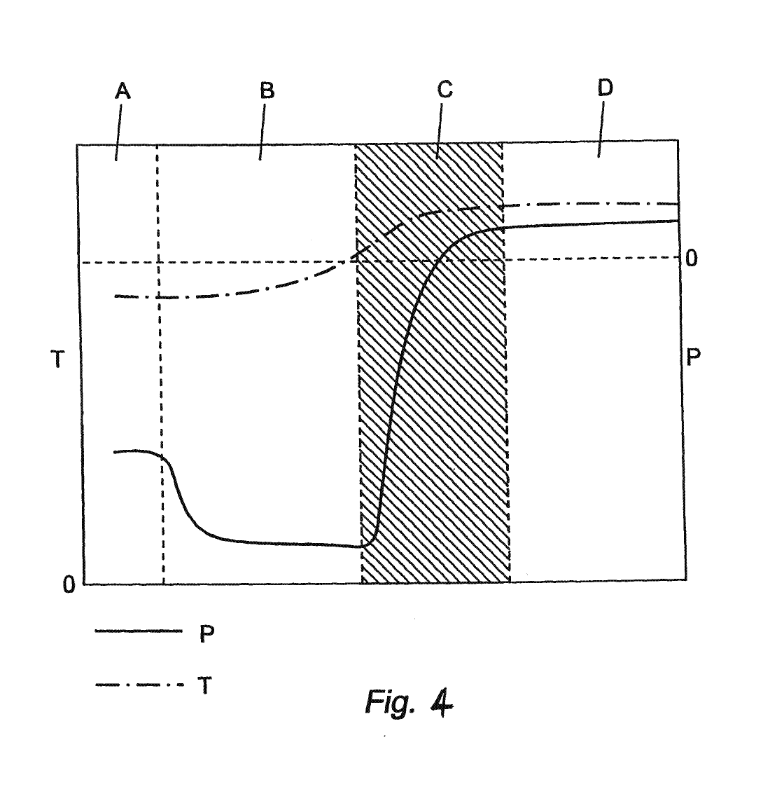

FIG. 4 is a graph showing pressure and temperature profiles of a working fluid slurry as it passes through the fluid processor;

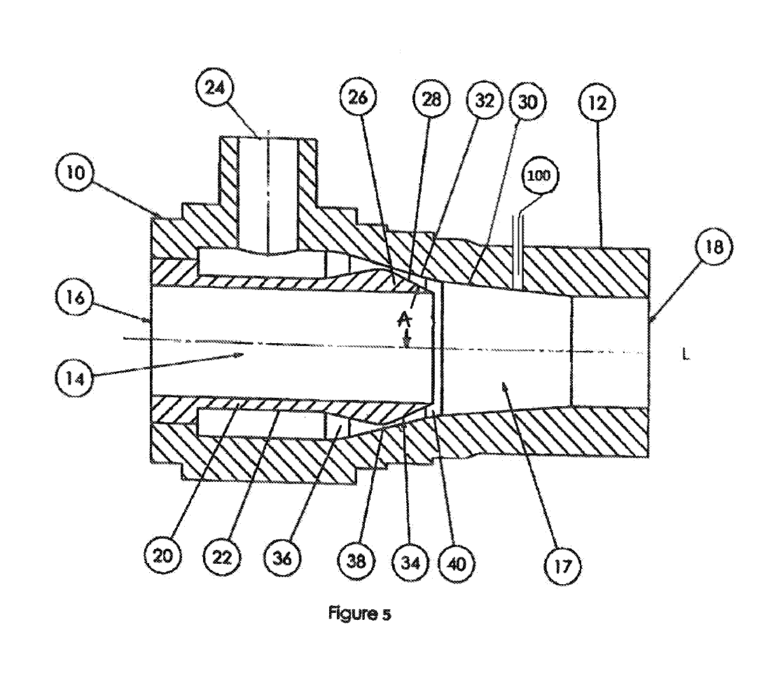

FIG. 5 is a cross sectional view of an alternative fluid processor;

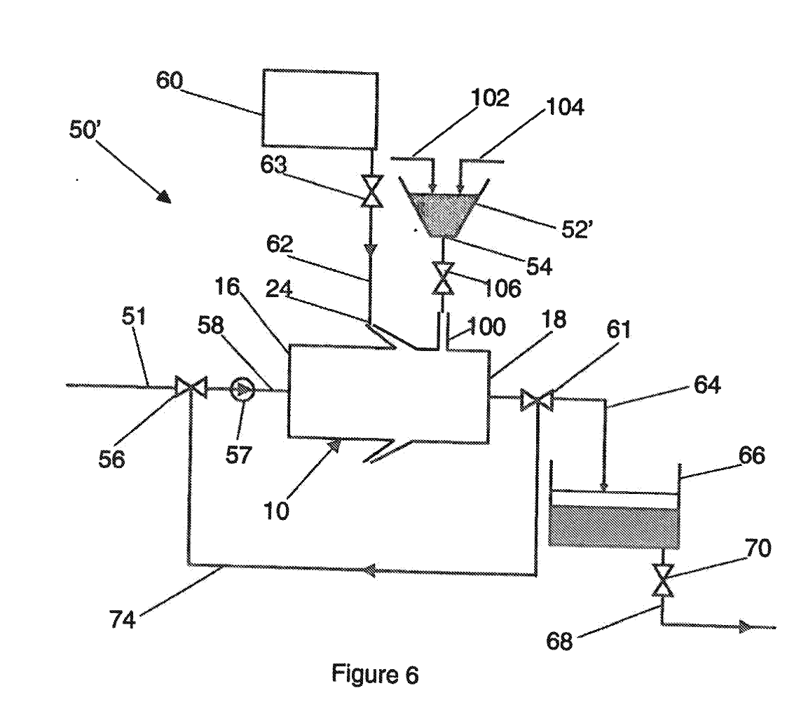

FIG. 6 is a schematic view of an alternative microorganism processing apparatus incorporating the fluid processor of FIG. 5; and

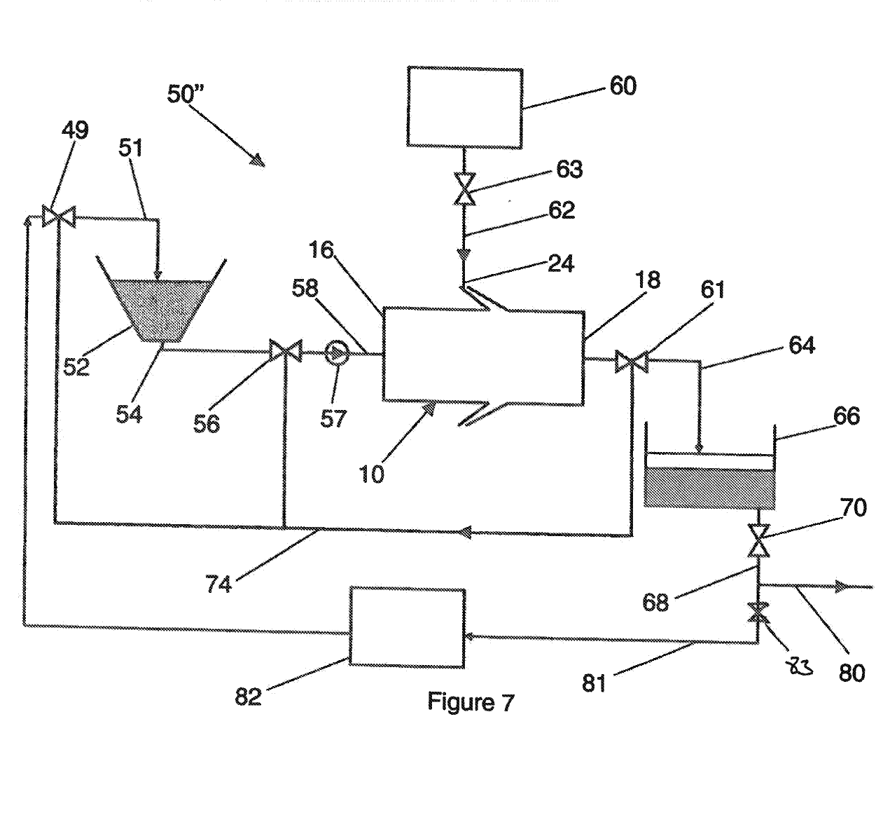

FIG. 7 is a schematic view of a further alternative microorganism processing apparatus based upon a modification to the apparatus shown in FIG. 3.

FIG. 1 is a vertical cross section through a fluid processor, generally designated 10. The processor 10 comprises a housing 12 within which is defined a longitudinally extending passage 14 with a longitudinal axis L. The passage has an inlet 16 and an outlet 18 and is of substantially constant circular cross section. The cross sectional area of the passage 14 is never less than that of the inlet 16, so that any solids that pass through the inlet 16 will not encounter any constraining area reduction that prevents their motion through the rest of the passage 14.

A protrusion 20 extends axially into the housing 12 from the inlet 16 and defines exteriorly thereof a plenum 22 for the introduction of a compressible transport fluid. The plenum 22 is provided with an inlet 24 which is connectable to a source of transport fluid (not shown in FIG. 1). The protrusion 20 defines internally thereof the inlet 16 and an upstream portion of the passage 14. The protrusion 20 has a distal end 26 remote from the inlet 16. The distal end 26 of the protrusion 20 has a thickness which increases and then reduces again so as to define an inwardly tapering surface 28. The housing 12 has a wall 30, which at a location adjacent that of the tapering surface 28 of the protrusion 20 is increasing in thickness. This increase in thickness provides a portion of the wall 30 with a surface 32 which has an inward taper corresponding to that of the tapering surface 28 of the protrusion 20. Between them the tapering surface 28 of the protrusion 20 and the tapering surface 32 of the wall 30 define an annular nozzle 34. The nozzle 34 has a nozzle inlet 36 in flow communication with the plenum 22, a nozzle outlet 40 opening into the passage 14, and a nozzle throat 38 intermediate the nozzle inlet 36 and the nozzle outlet 40. The nozzle 34 is a convergent-divergent nozzle. As will be understood by the skilled reader, this type of nozzle has a nozzle throat 38 having a cross sectional area which is less than that of both the nozzle inlet 36 and the nozzle outlet 40. There is a smooth and continuous decrease in cross-sectional area from the nozzle inlet 36 to the nozzle throat 38 and a smooth and continuous increase in cross-sectional area from the nozzle throat 38 to the nozzle outlet 40. By “smooth” it is meant that the nozzle 34 has no sudden step change or jump in cross-sectional area, though the surface might have a roughness, or small protuberances (vortex generators) to generate turbulence in the flow passing through the nozzle 34. The passage 14 also includes a mixing region 17, which is located in the passage immediately downstream of the nozzle outlet 40.

As an example the decrease and increase in the cross-sectional area of the nozzle 34 can be linear, or may have a more complex profile. One such profile might be that the stream-wise cross-section is substantially the same as that of a De Laval nozzle, which has a cross-section of an hour-glass-type shape.

Given that the nozzle 34 is annular, ensuring that the cross-sectional area varies in the appropriate manner requires the calculation of an equivalent angle of expansion for the nozzle 34. FIG. 2 shows this schematically. E1 is the radius of a circle having the same cross sectional area as the nozzle throat 38. E2 is the radius of a circle having the same cross sectional area as the nozzle outlet 40. The distance d is the equivalent path distance between the throat 38 and the outlet 40. An angle β is calculated by drawing a line through the uppermost points of E2 and E1 which intersects a continuation of the equivalent distance line d. This angle β can either be measured from a scale drawing or else calculated from trigonometry using the radii E1, E2 and the distance d. The equivalent angle of expansion γ for the transport fluid nozzle can then be calculated by multiplying the angle β by a factor of two, where γ=2β. The optimal expansion in cross sectional area of the annular nozzle has been achieved using an equivalent angle of expansion in the range 8 to 30 degrees.

Referring back to FIG. 1, an angle A is defined between the inner surface 28 of the transport nozzle outlet 40 and the longitudinal axis L of the passage 14. The angle formed between the inner surface 28 of the nozzle outlet 40 and the longitudinal axis L is constrained by the required equivalent angle of expansion γ and hence the cross-sectional area of the nozzle outlet 40. The angle A is preferably between 1 and 70 degrees to the longitudinal axis L, and most preferably between 15 and 35 degrees to the longitudinal axis L.

The resulting nozzle 34 is a convergent-divergent nozzle as described above. The average flow velocity of the transport fluid at any given cross-section along such a nozzle depends on the flow conditions (temperature, pressure, density, phase and, in the case of steam, on the dryness fraction) and on the cross-sectional area of the nozzle at that point. Under some flow conditions the transport fluid passing through such a nozzle 34 can be at subsonic velocities along its entire length, whilst at other flow conditions the fluid can undergo first subsonic and then supersonic flow as it passes along the nozzle length, up to and including fluid that is at supersonic velocities throughout the entire divergent portion of the nozzle and even downstream of the nozzle exit. Such flow conditions can be controlled by, for instance, a pressure controller at the transport fluid source or transport fluid nozzle inlet 24, or at some point between the two. As an example, a control valve (not shown) may be located immediately before the nozzle inlet 24. A pressure tapping may be located between the valve and the plenum 22 and linked to a pressure measuring device (not shown). An operator can adjust the valve such that it constricts transport fluid flow to a greater or lesser extent in order that the pressure in this region is maintained at a desired level or within a desired range. In a process plant, a remote controller is linked to the pressure measuring device such that the controller automatically opens or closes the valve so as to maintain the pressure at the predetermined level or within the desired range.

FIG. 3 shows schematically an apparatus for processing microorganisms in order to recover intracellular material therefrom. In the context of the present invention an intracellular material is an oil, a chemical compound, a protein compound or pharmaceutical compound contained within the cells of the microorganisms. Whilst the preferred embodiment of the process described here is primarily concerned with recovering oils from the microorganisms, recovery of chemical and pharmaceutical compounds such as the oil-soluble pigments lycopene and beta-carotene is also possible with the present invention. The apparatus 50 comprises a fluid processor 10 of the type shown in FIG. 1 and a mixing vessel, or hopper, 52 into which in this exemplary embodiment an algae culture (e.g. algae in water; dried algae) is added. A diluent, or working fluid, such as water is added to the hopper 52 via a supply line 51 so to form a working fluid slurry, or algal working fluid, containing an appropriate concentration of algal cells. The concentration of algal cells in the working fluid may be between 0.1 and 18 percent weight for weight. The hopper 52 has an agitator (not shown) for stirring and/or mixing its contents, as well as an outlet 54 controlled by an outlet valve 56.

Downstream of the hopper 52 is the fluid processor 10. The outlet 54 of the hopper 52 is fluidly connected to the inlet 16 of the passage 14 shown in FIG. 1 via a first processing line 58. Also shown in FIG. 3 is a transport fluid supply 60, which is connected to the plenum inlet 24 of the processor 10 via a transport fluid supply line 62. A supply valve 63 controls flow of the transport fluid from the supply 60. Downstream of the processor 10 is a processing vessel 66. As will be explained in more detail below, the processing vessel 66 can either act as a separation tank for separating the oil released from the algae during the processing, or else it can act as a holding tank in which further treatment of the algae can be carried out. The processing vessel 66 is fed via a second processing line 64 fluidly connected to the outlet 18 of the processor 10. The processing vessel 66 has at least one drain line 68 which is controlled by a drain valve 70.

If necessary, a pump 57 may be provided on the first processing line 58 to pump the algal working fluid from the hopper 52 into the passage 14. When present, the pump 57 is preferably a progressive cavity pump, also known as a rotary positive displacement pump.

The apparatus may further comprise a recirculation loop 74 fluidly connecting the second processing line 64 downstream of the fluid processor(s) with the first processing line 58, the hopper 52 or the supply line 51 upstream of the fluid processor(s). Suitable diverter valves 49,61 can be placed in the supply line 51 (as shown) or first processing line 58 and the second processing line 64 in order to selectively divert the fluid flow through the recirculation loop 74. The loop may also include a recirculation pump (not shown) to assist in returning the flow to the hopper or first processing line.

The various valves in the apparatus, as well as the pump if present, may be controlled by a programmable system controller 90.

The process carried out by the apparatus 50 will now be described. Initially, a suitable microorganism culture such as algae, for example, is introduced into the hopper 52. If the algae is not already in water or another suitable fluid it can be mixed with a diluent or working fluid via supply line 51 so as to form an algal working fluid or working fluid slurry in the hopper 52, having an appropriate concentration of algae to working fluid.

When it is time for processing to commence the outlet valve 56 is opened in order to allow the algal working fluid to flow along the first processing line 58 into the processor 10. When present, the pump 57 is started to assist with the flow. The supply valve 63 controlling the supply of transport fluid to the processor 10 is also opened. Consequently, transport fluid flows from the transport fluid supply 60 into the processor 10 via the plenum 22. In this preferred embodiment, the transport fluid is a compressible gas which is heated in the transport fluid supply 60. The gas is preferably steam and the transport fluid supply 60 is preferably a steam generator.

Referring to FIG. 1, the convergent divergent shape of the nozzle 34 accelerates the transport fluid and a high velocity, preferably supersonic, jet of transport fluid is injected into the fluid passage 14 from the nozzle outlet 40. At the same time, the algal working fluid is flowing through the inlet 16 of the passage 14. As the transport fluid is injected into the passage 14 from the nozzle 34 it imparts a shearing force on the working fluid as it passes the nozzle outlet 40. This shearing force atomizes the working fluid and breaks down the cellular structure of the algae contained therein. The differences in velocity, temperature and pressure between the transport fluid and the algal working fluid also leads to a momentum transfer from the high velocity transport fluid to the lower velocity working fluid, causing the working fluid and algal cells therein to accelerate. This acceleration of the algal cells creates a pressure differential across the cells and/or between the internal cell and external environment, which also assists in breaking them down.

The effects of the process on the temperature and pressure of the algal working fluid can be seen in the graph of FIG. 4, which shows an example of the profile of the temperature and pressure as the working fluid passes through various points in the passage 14 of the fluid processor 10 of FIG. 1. The graph has been divided into four sections A-D, which correspond to various sections of the passage 14 shown in FIG. 1. Section A corresponds to the section of the passage 14 between the inlet 16 and the nozzle 34. Section B corresponds to the upstream section of the mixing region 17 extending downstream from the nozzle outlet 40 to an intermediate portion of the mixing region 17. Section C corresponds to a downstream section of the mixing region 17 extending between the aforementioned intermediate portion of the region 17 and the outlet 18, while section D illustrates the temperature and pressure of the algal working fluid as it passes through the outlet 18.

The transport fluid is injected into the algal working fluid at the beginning of section B of the FIG. 4 graph. The velocity of the transport fluid, which is preferably supersonic at the point of injection, and its expansion upon exiting the nozzle 34 cause an immediate pressure reduction. A dispersed phase of working fluid droplets in a continuous vapour phase of transport fluid (also known as a vapour-droplet flow regime) is created in the passage 14 and flows towards the outlet 18. As it moves towards the outlet 18 the fluid flow will begin to decelerate. This deceleration will result in an increase in pressure within the mixing region 17. At a certain point within the mixing region 17, the decrease in velocity and rise in pressure will result in a rapid condensation of the vapour in the vapour-droplet regime. The point in the mixing region 17 at which this rapid condensation begins defines a condensation shockwave within the passage 14. A rise in pressure and consequent vapour-to-liquid phase change takes place across the condensation shockwave as shown in section C of the FIG. 4 graph, with the flow returning to the liquid phase on the downstream side of the shockwave illustrated by section D of the graph. The dotted line across the graph shows zero gauge pressure, i.e. anything under the line is a negative pressure or vacuum whilst anything above the line is a positive pressure. Alternatively it can be understood that if the entire system is pressurised then this graph would show a relative negative pressure, or vacuum, compared with system pressure and not an absolute negative pressure. The position of the shockwave within the passage 14 is determined by the supply parameters (e.g. pressure, density, velocity, temperature) of the transport fluid and of the algal working fluid, the geometry of the fluid processor, and the rate of heat and mass transfer between the transport and working fluids.

As previously stated, the shear force applied to the working fluid and the subsequent turbulent flow created by the injected transport fluid disrupts the cellular structure of the algae contained in the algal working fluid. As the working fluid passes through the low pressure area and subsequent condensation shockwave formed in the passage 14, the algae are further disrupted by the sudden changes in pressure occurring, as illustrated by the pressure profile in sections B and C of FIG. 4.

Referring back to FIG. 1, the angle A at which the transport fluid exits the nozzle 34 affects the degree of shear between it and the algal working fluid passing through the passage 14 as well as the turbulence levels in the vapour-droplet flow regime created following the atomization of the fluid content.

A cavitation process may also take place within the mixing region 17 due to the vaporization and subsequent rapid condensation of the working fluid droplets. Cavitation creates temporary, localised high temperatures and pressures that can also assist in the break up of the algal cells.

Referring back to FIG. 3, downstream of the mixing region 17 in the fluid processor 10 the condensed working fluid, algae and oil and/or other intracellular material released from the algae due to the aforementioned cellular disruption leave the processor 10 via outlet 18. They are then carried via the second processing line 64 to the processing vessel 66. The processing vessel 66 can act as a gravity-assisted separation vessel where the intracellular material, the residual matter from the algae and the working fluid can be left to separate from one another under gravity. The separation vessel may include a centrifuge to assist with the separation. The separated constituents can then be retrieved from the surface of the fluid or else drained one at a time from the vessel 66 via the one or more drain lines 68 when the respective drain valve 70 is opened, or else they can continue downstream for further processing at a subsequent stage in a processing plant. The working fluid recovered from the processing vessel 66 can be re-used in the process by being returned to the mixing vessel/hopper 52.

In some instances, the disruption to the cellular structure of the algae will not result in the immediate release of the oil and/or other intracellular material held therein. However, the cellular disruption will at very least increase the porosity of the cell walls. In this case, the processing vessel 66 may be utilised as a further treatment tank, where one or more additives (e.g. solvents) can be introduced into the algal working fluid in order to work on the algae through these porous cell walls. Given that the passage through the fluid processor 10 has increased the porosity of the cell walls, much less additive will be required to ensure the release of the intracellular material held in the algae than would be needed without the “pre-treatment” by the fluid processor. After the release and separation of the oil the algal cells can be returned to a growth container or facility.

An alternative embodiment of fluid processor and associated microorganism processing apparatus are shown in FIGS. 5 and 6.

Referring firstly to FIG. 5, the fluid processor 10′ has substantially the same components and internal geometry as the fluid processor 10. Consequently, the same reference numbers are used in both embodiments in order to indicate common elements in each fluid processor. Those common elements will not be described in detail again here. Where the alternative embodiment differs from the FIG. 1 embodiment is that an entrainment port 100 is provided, which opens into the mixing region 17 of the passage 14 downstream of the nozzle outlet 40.

Referring to FIG. 6, it can be seen that the entrainment port 100 is connected to a mixing vessel, or hopper, 52′ forming part of the alternative apparatus 50′. As with the alternative fluid processor, the same reference numbers are used in both embodiments of the apparatus in order to indicate common elements, which will not be described in detail again here.

From FIG. 6 it can thus be seen that in the alternative apparatus 50′ the outlet 54 of the hopper 52′ is no longer in fluid communication with the inlet 16 of the fluid processor passage 14, but is instead in fluid communication with the entrainment port 100. The hopper 52′ has first and second supply lines 102,104 which supply a diluent, or working fluid, and a microorganism culture (e.g. algae in water; dried algae), respectively, into the hopper 52′. An outlet valve 106 controls the flow of the hopper contents to the entrainment port 100.

The process carried out by the alternative apparatus 50′ will now be described. Unless specifically stated otherwise the steps of the alternative process are the same as those of the first process described above.

Initially, a suitable microorganism culture such as an algae is introduced into the hopper 52′ via the second supply line 104. If the algae is not already in water or another suitable fluid it can be mixed with a diluent or working fluid via the first supply line 102 so as to form an algal working fluid, or working fluid slurry, in the hopper 52′ having an appropriate concentration of algae to working fluid.

When it is time for processing to commence the outlet valve 56 is opened in order to allow a process fluid to flow along the first processing line 58 from a process supply 51 into the processor 10′. The process fluid may be identical to the working fluid which is mixed with the algae culture in the hopper 52′. When present, the pump 57 is started to assist with the flow of the process fluid into the processor 10′. The supply valve 63 controlling the supply of transport fluid to the processor 10′ is also opened. Consequently, transport fluid flows from the transport fluid supply 60 into the processor 10′ via the plenum 22.

As in the first embodiment the injection of the transport fluid into the passage 14 from the nozzle 34 imparts a shearing force on the process fluid as it passes the nozzle outlet 40. This shearing force atomizes the process fluid and creates a dispersed phase of process fluid droplets within a continuous vapour phase of transport fluid. As highlighted in the FIG. 4 graph of pressure and temperature, the velocity of the transport fluid and its expansion upon exiting the nozzle 34 cause an immediate pressure reduction within the mixing region 17 of the passage 14. By opening the supply valve 106, the algal working fluid from the hopper 52′ enters this low pressure region via the entrainment port 100, and is mixed with the dispersed process fluid.

As it moves towards the outlet 18 the combined flow of process and algal working fluids will begin to decelerate. This deceleration will result in an increase in pressure within the downstream portion of the mixing region 17. At a certain point within the mixing region 17, the decrease in velocity and rise in pressure will result in a rapid condensation of the vapour phase. The point in the mixing region 17 at which this rapid condensation begins defines a condensation shockwave within the passage 14.

As with the first process, the shear force applied and the subsequent turbulent flow created by the injected transport fluid disrupts the cellular structure of the algae contained in the algal working fluid entering the passage 14 through the entrainment port 100. As the working fluid passes through the low pressure area and subsequent condensation shockwave formed in the passage 14, the algae are further disrupted by the sudden changes in pressure occurring, as illustrated by the pressure profile in sections B and C of FIG. 4.

Referring back to FIG. 6, the condensed fluids, algae and oil and/or other intracellular material released from the algae due to the aforementioned cellular disruption are processed downstream of the processor 10′ in the same manner as in the first embodiment. As in the first embodiment, the various valves and pumps may be controlled by a programmable system controller (not shown in FIG. 6).

A further alternative embodiment of the apparatus is shown in FIG. 7. This embodiment is based upon a modification to the apparatus shown in FIG. 3. The majority of components in the FIG. 7 embodiment are shared with the FIG. 3 embodiment. Those components share reference numerals and will therefore not be described again here.

The modification in the FIG. 7 embodiment is that a further recirculation loop 81 is provided at the downstream end of the apparatus. In this case the loop 81 is in communication with a processing line 80 leading downstream from the drain line 68. The loop 81 includes a control valve 83 which selectively allows fluid to enter the loop 81 from the processing line 80. The loop also includes an algae growth vessel 82, which receives the fluid returning through the loop 81. As in the previously described embodiments, the various valves and pumps may be controlled by a programmable system controller (not shown in FIG. 7).

In this embodiment, the apparatus is adapted such that disruption of the cellular structure of the algae or other microorganism is limited, whereby oils may be extracted from the microorganisms but the cellular structure remains intact. In this example, the fluid returning via loop 81 may contain live algae cells which are returned to the algae growth vessel 82. This process is sometimes referred to as “milking”, which can release extracellular oils from between the cells that have formed clumps and have oil suspended between them.

The apparatus and associated methods of the present invention allow the oils and other intracellular material present in microorganisms to be released and recovered with a significant reduction in the amount of chemical additive needed. In some instances a complete disruption of the cellular structure of the organism will occur using the present invention, thereby removing the need for any additive at all. As these additives can be dangerous to handle and/or expensive, it is to the benefit of the processor if there is a significant reduction in the amount used or indeed no need to use them at all. In other instances, only a minimal disruption of cell structure will occur, this will allow the extraction of oils whilst preserving cell viability for future biosynthesis of oil and/or other valuable products.

As the present invention is capable of releasing the intracellular material from the microrganisms in a single stage process, the present invention also has a number of advantages over existing two stage processes where drying and cold pressing of the microorganisms is necessary following a chemical pre-treatment phase. Releasing the intracellular material in a single stage reduces processing time as well as energy requirements. In addition, the reduction or removal of chemical additives from the process achieves a corresponding reduction or removal of any environmental clean-up processes once the oil and/or other intracellular material has been released and recovered from the apparatus.

The entrainment port in the second embodiment of the processor and apparatus allows the working fluid slurry to be entrained directly into the mixing region of the processor passage, where it is mixed with the process fluid. Entraining the slurry directly in this manner allows the microorganisms to be exposed to additional process conditions which further enhance the extraction of the intracellular components and/or degradation of the cell walls. For example, variations in osmotic potential or temperature between the process fluid and slurry can expose the cells to supplemental physiological or physical shock as the slurry is entrained into the mixing region.

In addition to a process flow pump, a further pump may be provided between the hopper and entrainment port so as to ensure a desired entrainment or flow rate. Where both the process fluid and slurry are pumped to the apparatus, the pressure applied by the transport fluid will result in a pressure being applied to the process fluid. In this situation, the transport fluid flow may be pulsed so that there is a cyclical pressurization and depressurization taking place within the apparatus, with a resultant generation and collapse of the dispersed droplet-vapour regime in concert with the pulsing of the transport fluid. This will create further physical stresses on the cells, still further enhancing the performance of the apparatus and process.

As with the process of the first embodiment the alternative processes may employ a recirculation loop, as shown in FIGS. 6 and 7, to continue processing of the fluids in a batch-type process. Alternatively, the apparatus may employ a number of fluid processors in series downstream of the main processor in order to obtain higher flow temperatures and/or to further mix the fluids before the next process step.

When the oil extraction is compatible with maintaining cell viability, a second recirculation loop may be used, as shown in FIG. 7. This loop returns extracted cells and/or aqueous fraction of the working fluid into the growth vessel or facility.

Where the processing of the present invention concerns marine algae the working fluid introduced in the hopper as a diluent may be salt water having a salt content greater than 50 per mille. Alternatively, the working fluid may have a salt content of between 1 and 50 per mille to encourage osmosis between the contents of the marine algae and their immediate environment. This osmosis will cause the cells to swell as they absorb water, placing a strain on the cell wall structure. Such swollen cells are even more likely to be disrupted when they pass through the low pressure area within the fluid processor.

Even though the present invention reduces the processing time required to release the intracellular material, the process may also comprise an initial step of introducing an additive (e.g. enzymes such as cellulases, alginate lyases or polygalacturonases) to the contents of the hopper in order to begin degrading of the cellular structure of the microorganisms prior to entering the fluid processor.

Whilst the apparatus described above utilise a single fluid processor, they may instead comprise a plurality of such processors arranged in series and/or parallel with one another to form a processing array.

The working fluid used in the process of the present invention is preferably water, with or without salt content. However, non-limiting examples of other suitable working fluids include hexane, decane, dodecane, n-methyl morpholine-n-oxide, chloroform, ethanol, organic solvents, and organosulphur solvents such as dimethyl sulphoxide (DMSO). These alternative working fluids may also be mixed with water, whether they are miscible or immiscible.

A further additive may be added to the algae in the hopper in order to alter the pH of the algal working fluid. Altering the pH of the algae can increase the likelihood of the cellular structure of the algae rupturing during the subsequent processing. The pH change can also contribute to the flocculation effect.

Any of the additives referred to in this specification could also be introduced to the slurry via an additive port in the fluid processor. The port may be connected to the passage in the processor to allow one or more additives to be added to the slurry in the passage. Preferably, the additive port is located in the passage immediately downstream of the nozzle outlet at the upstream end of the mixing region, or immediately downstream of the entrainment port in the case of the second embodiment of the fluid processor.

The transport fluid utilised in the process of the present invention is preferably steam. However, non-limiting examples of other suitable transport fluids are carbon dioxide and nitrogen.

Carbon dioxide or an alternative compressed gas such as nitrogen, for example, may be injected into the slurry in the hopper via a gas injector, whereby it is absorbed by the microorganisms present. Subsequently passing the microrganisms through the pressure variations in the fluid processor will cause a rapid expansion of this gas, again assisting in the disruption of the cell walls. To further assist this gas expansion the apparatus may further comprise a first pressure-regulating valve upstream of the fluid processor to maintain a predetermined pressure in the first supply line and hopper. A second pressure-regulating valve may be located downstream of the fluid processor. The compressed gas may then be recovered, scrubbed if necessary and re-used.

The preferred methods described can be conducted at a range of temperatures dependent on the method of oil extraction used. For example, when extracting extracellular oils it is preferable to keep the temperatures below 50° C. Destructive extraction can take place at any temperature, but preferably between 5° C. and 150° C. and most preferably between 50° C. and 150° C.

These and other modifications and improvements may be incorporated without departing from the scope of the invention.

1. A method of processing microorganisms, comprising:
mixing microorganisms with a working fluid to form an working fluid slurry; and
injecting a transport fluid through a transport fluid nozzle into the working fluid slurry in order to disrupt the cellular structure of the microorganisms.

2. The method of claim 1, wherein the microorganisms are algae.

3. The method of claim 1, further comprising the steps of: supplying the working fluid slurry to a fluid processor passage having an inlet and an outlet, wherein the cross sectional area of the passage between the inlet and the outlet does not reduce below the cross sectional area at the inlet;
supplying the transport fluid from a transport fluid source to the transport fluid nozzle which circumscribes the passage and opens into the passage intermediate the inlet and the outlet, the transport fluid nozzle having a nozzle inlet, a nozzle outlet, and a nozzle throat intermediate the nozzle inlet and nozzle outlet which has a cross sectional area which is less than that of both the nozzle inlet and nozzle outlet; and
accelerating the transport fluid through the transport fluid nozzle so as to inject the transport fluid into the working fluid slurry.

4. The method of claim 3, further comprising the step of recovering any intracellular material released by the microorganisms downstream of the fluid processor.

5. The method of claim 4, wherein the intracellular material is oil.

6. The method of claim 4, wherein the recovery step includes separating the intracellular material from the working fluid slurry in a separation vessel.

7. The method of claim 4, wherein the recovery step includes adding an additive to the working fluid slurry to encourage the release of the intracellular material.

8. The method of claim 7, wherein the additive includes a flocculant for the concentration and separation of the intracellular material from the rest of the working fluid.

9. The method of claim 4, wherein the recovery step includes adding demulsifiers to the working fluid slurry to facilitate separation of an oil fraction from an aqueous fraction.

10. The method of claim 1, wherein the working fluid is water.

11. The method of claim 10, wherein the water has a salt content of between 1 and 50 per mille.

12. The method of claim 1, wherein the working fluid is selected from the group consisting of hexane, n-methyl morpholine n-oxide, dodecane, dichloromethane, chloroform, ethanol and dimethyl sulfoxide.

13. The method of claim 1, wherein the mixing step includes the addition of one or more degrading additives to chemically degrade the cellular structure of the microorganisms.

14. The method of claim 13, wherein one degrading additive is an enzyme to enzymatically degrade the cellular structure of the microorganisms.

15. The method of claim 1, wherein the mixing step includes the addition of one or more pH-altering additives to alter the pH of the working fluid slurry.

16. The method of claim 3, wherein the transport fluid is steam and the transport fluid source is a steam generator.

17. The method of claim 3, further comprising the steps of:
injecting a compressed gas into the working fluid slurry prior to the step of supplying the fluid to the fluid processor passage; and
holding the working fluid slurry under pressure upstream of the fluid processor.

18. The method of claim 17, wherein the compressed gas is selected from the group consisting of carbon dioxide, nitrogen and air.

19. The method of claim 3, wherein the working fluid slurry is supplied via an entrainment port which opens into the passage downstream of the nozzle outlet.

20. The method of claim 19, further comprising the step of supplying a process fluid to the inlet of the passage.

21. The method of claim 20, wherein the process fluid is water.

22. The method of claim 21, wherein the water has a salt content of between 1 and 50 per mille.

23. The method of claim 20, wherein the process fluid is selected from the group consisting of hexane, decane, dichloromethane, n-methyl morpholine n-oxide, chloroform, ethanol, and dimethyl sulfoxide.

24. The method of claim 20, wherein the process fluid and working fluid slurry have different osmotic potentials and/or temperatures.

25. The method of claim 3, wherein the supply and subsequent injection of the transport fluid is pulsed.

26. The method of claim 3, further comprising the step of returning fluid flow from downstream of the passage outlet to the inlet of the passage via a first return loop and diverter valve.

27. The method of claim 3, further comprising the step of returning fluid flow from downstream of the passage outlet to a growth vessel via a second return loop and diverter valve.

28. The method of claim 27, wherein the working fluid slurry returned to the growth vessel contains live microorganisms.

29. An apparatus for processing microorganisms, the apparatus comprising:
a mixing vessel adapted to receive and mix supplies of microorganisms and a working fluid to form a working fluid slurry; and
a transport fluid nozzle adapted to inject a transport fluid into the working fluid slurry in order to disrupt the cellular structure of the microorganisms.

30. The apparatus of claim 29, further comprising:
a fluid processor including the transport fluid nozzle and a passage having an inlet and an outlet; and
a transport fluid source in fluid communication with the transport fluid nozzle; wherein the transport fluid nozzle circumscribes the passage and opens into the passage intermediate the inlet and outlet, the mixing vessel is in fluid communication with the passage, the cross sectional area of the passage between the inlet and outlet does not reduce below the cross sectional area at the inlet, and the transport fluid nozzle is a convergent-divergent nozzle having a nozzle inlet, a nozzle throat, and a nozzle outlet, and the cross sectional area of the nozzle throat is less than that of both the nozzle inlet and nozzle outlet.

31. The apparatus of claim 30, wherein the mixing vessel is in fluid communication with the inlet of the passage.

32. The apparatus of claim 30, wherein the processor further comprises an entrainment port opening into the passage downstream of the nozzle outlet, and wherein the mixing vessel is in fluid communication with the entrainment port.

33. The apparatus claim 30, wherein the transport fluid source is a steam generator.

34. The apparatus of claim 30, wherein the transport fluid source includes a transport fluid pressure controller.

35. The apparatus of claim 30, wherein the transport fluid source is adapted so as to pulse the supply of transport fluid.

36. The apparatus of claim 30, further comprising a plurality of fluid processors connected to one another in series and/or parallel.

37. The apparatus of claim 30, further comprising a separation vessel in fluid communication with the outlet of the passage.

38. The apparatus of claim 30, wherein the transport fluid nozzle has an equivalent angle of expansion from the nozzle throat to nozzle outlet of between 8 and 30 degrees.

39. The apparatus of claim 30, wherein the fluid processor includes a housing and a protrusion which extends axially into the housing, whereby the protrusion defines a portion of the passage downstream of the passage inlet and an inner surface of the transport fluid nozzle outlet.

40. The apparatus of claim 39, wherein the passage has a longitudinal axis, and the inner surface of the transport fluid nozzle outlet is at a maximum angle of 70 degrees relative to the longitudinal axis.

41. The apparatus of claim 40, wherein the inner surface of the transport fluid nozzle outlet is at an angle of between 15 and 35 degrees relative to the longitudinal axis.

42. The apparatus of claim 30, further comprising a progressive cavity pump adapted to pump working fluid slurry into the fluid processor passage.

43. The apparatus of claim 30, further comprising a first return loop and diverter valve downstream of the passage outlet, the first return loop and diverter valve adapted to return fluid flow to the inlet of the passage.

44. The apparatus of claim 43, further comprising a growth vessel, and a second return loop and diverter valve adapted to return fluid flow from the processing vessel to the growth vessel.

45. The apparatus of claim 44, wherein the second return loop diverts the working fluid slurry from downstream of the passage outlet back to the growth container.

46. The apparatus of claim 30, wherein the mixing vessel comprises a gas injector adapted to inject a compressed gas into the vessel.

47. The apparatus of claim 46, further comprising a first pressure regulating valve adapted to maintain a predetermined pressure upstream of the fluid processor.

48. The apparatus of claim 47, further comprising a second pressure regulating valve adapted to maintain a predetermined pressure downstream of the fluid processor.

49. The apparatus of claim 30, further comprising one or more flow control valves and a programmable system controller adapted to selectively activate the one or more control valves.
